(19) United States
(12) Patent Application Publication
(10) Pub. No.: US 2020/0179898 A1
(43) Pub. Date: Jun. 11, 2020

(54) MICROWAVE REACTOR AND MANUFACTURING METHOD OF BIODIESEL

(71) Applicant: NATIONAL TSING HUA UNIVERSITY, Hsinchu (TW)

(72) Inventors: Tsun-Hsu CHANG, Hsinchu City (TW); Hsien-Wen CHAO, Taoyuan City (TW); Shen-Fu CHEN, Changhua County (TW)

(21) Appl. No.: 16/529,731

(22) Filed: Aug. 1, 2019

(30) Foreign Application Priority Data
Dec. 6, 2018 (TW) 107143974

(51) Int. Cl. B01J 19/12; C10L 1/02; C07C 67/03

(52) U.S. Cl. CPC B01J 19/126; B01J 2219/1943; C07C 67/03; C10L 2200/0476; C10L 2290/18; C10L 2290/36; C10L 2290/544; C10L 2290/56; B01J 2219/0877; B01J 2219/0871; B01J 2219/1215; B01J 2219/1239; B01J 2219/126; B01J 2219/1296; B01J 2219/185; C10L 1/02

(57) ABSTRACT

A microwave reactor includes a chamber, at least one microwave source, a sprayer and a vapor extractor. The chamber includes a containing space and a reacting space. The containing space is communicated with the reacting space and provided for containing a reactant. The microwave source is connected to one side wall of the reacting space of the chamber. The sprayer is communicated with the containing space of the chamber for turning the reactant into a mist and spraying the mist in the reacting space of the chamber. The vapor extractor is connected to the reacting space. When the water contained in the mist is gasified to produces a water vapor, the water vapor can be exhausted from the chamber by the vapor extractor.

### RELATED APPLICATIONS

This application claims priority to Taiwan Application Serial Number 107143974, filed Dec. 6, 2018, which is herein incorporated by reference.

### BACKGROUND

### Technical Field

The present disclosure relates to a microwave reactor and a manufacturing method of biodiesel. More particularly, the present disclosure relates to a microwave reactor for heating to promote a chemical reaction or complete the reaction at a lower temperature and a manufacturing method of biodiesel.

### Description of Related Art

Microwaves can pass through materials and cause polar molecules to start oscillating so as to heat them up uniformly. Many studies also prove that the microwaves can raise a reaction rate of a chemical reaction so that the microwaves have been broadly applied in various industries recently.

However, when the microwaves are applied in a large chamber for performing a heating process, they are always absorbed by an outer portion of the reactant due to a larger volume of the reactant. Thus, the microwaves cannot penetrate to an inner portion of the reactant so as to generate hot-pots in the reactant, that is, the reactant cannot be heated uniformly. Accordingly, it is hard to control the reaction rate as well as the quality of products.

Moreover, an electrical discharge is easily happened if there is a metal inside a microwave chamber. It will cause higher dangerous in a close chamber so that a metal stirring bar, which is commonly applied in a reaction chamber, is not suitable for the microwave chamber.

### SUMMARY

According to one aspect of the present disclosure, a microwave reactor, which includes a chamber, at least one microwave source, a sprayer and a vapor extractor, is provided. The chamber includes a containing space and a reacting space, wherein the containing space is communicated with the reacting space and for containing a reactant therein. The microwave source is connected to one side wall of the reacting space of the chamber. The sprayer is provided for turning the reactant into a mist and spraying the mist in the reacting space. The vapor extractor is connected to the reacting space. When the mist is heated and a water contained is gasified to produce a water vapor, the water vapor can be exhausted from the chamber by the vapor extractor.

According to another aspect of the present disclosure, a manufacturing method of a biodiesel including following steps is provided. A mix of Glyceryl ester and methanol to be reacted is a mist through a sprayer at first, and the mist is sprayed in a reacting space. The mist is then heated by at turned into least one heating source so as to gasify a water contained for producing a water vapor. The water vapor can be extracted by a vapor extractor, and a generated methyl ester is obtained.

### BRIEF DESCRIPTION OF THE DRAWINGS

The present disclosure can be more fully understood by reading the following detailed description of the embodiment, with reference made to the accompanying drawings as follows:

FIG. 1 is a schematic view of a microwave reactor according to one embodiment of the present disclosure.

FIG. 2 is a cross-sectional view along line 2-2 of the microwave reactor shown in FIG. 1.

FIG. 3 is a schematic view of a microwave reactor according to another embodiment of the present disclosure.

FIG. 4 is a flow chart of a manufacturing method of biodiesel according to yet another embodiment of the present disclosure.

### DETAILED DESCRIPTION

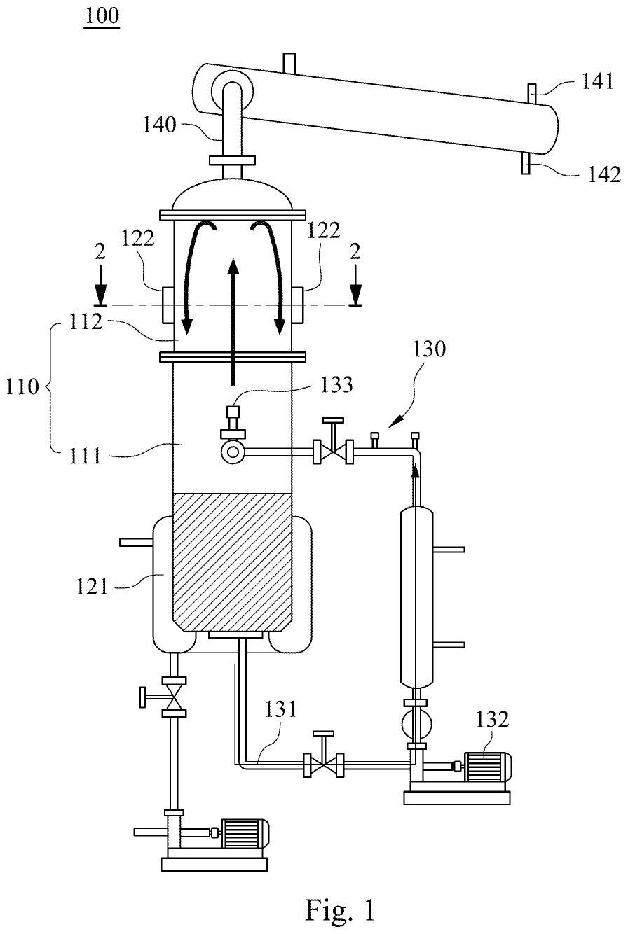

FIG. 1 is a schematic view of a microwave reactor 100 according to one embodiment of the present disclosure. In FIG. 1, the microwave reactor 100 includes a chamber 110, at least one microwave source 122, a sprayer 130 and a vapor extractor 140.

The chamber 110 includes a containing space 111 and a reacting space 112, wherein the containing space 111 is communicated with the reacting space 112 and for containing a reactant therein.

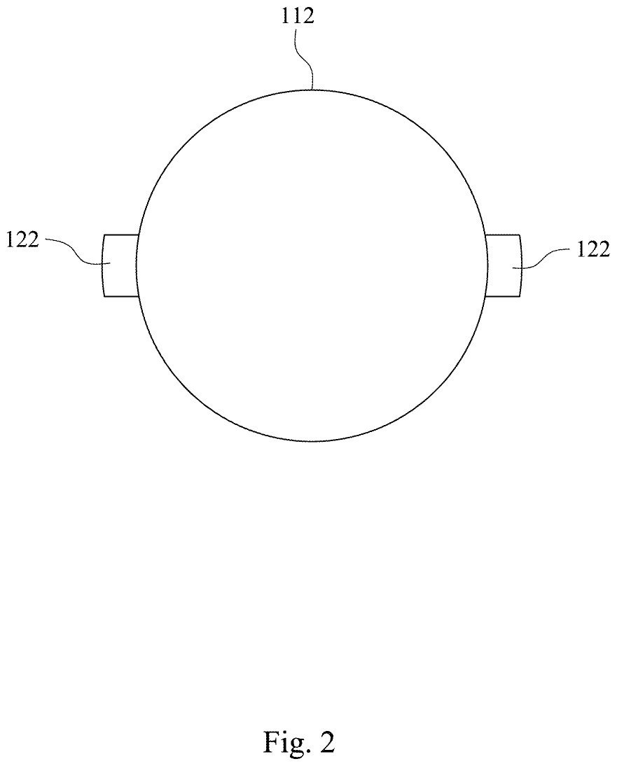

The microwave source 122 is connected to one side wall of the reacting space 112 of the chamber 100. Please refer to FIG. 2, which is a cross-sectional view along line 2-2 of the microwave reactor 100 shown in FIG. 1. In FIG. 2, there are two microwave sources 122 correspondingly disposed on the side wall of the reacting space 112. That is, the two microwave sources 122 are located at the same heights of the side wall of the reacting space 112. It is to be noted that the microwave reactor 100 can include a plurality of the microwave sources 122, such as three, four or more, and the present disclosure is not limited thereto.

The sprayer 130 is communicated with the containing space of the chamber and for turning the reactant into a mist and spraying the mist in the reacting space 112.

The vapor extractor 140 is connected to the reacting space 112. When the water contained in the mist is gasified in the reacting space 112 to produce a water vapor, the water vapor can be exhausted from the chamber 110 by the vapor extractor 140. In details, the vapor extractor 140 includes a vacuum pump 141 and a condensed water outlet 142. The water vapor can be extracted by using the vacuum pump 141 and further directed outward through the condensed water outlet 142. Moreover, the water vapor also can be produced by gasifying water generated after the mist undergoes a chemical reaction, and the present disclosure is not be limited thereto.

When the microwave reactor 100 is applied to a manufacturing process of a biodiesel, the chamber 110 is comparatively large. Thus, the microwaves cannot penetrate the reactant when the reactant in the containing space 111 of the chamber 110 is directly heated without being sprayed into mist. It is unfavorable for controlling the reaction rate and the quality of the biodiesel. Thus, after the reactant is atomized and spayed in the reacting space 112 through the sprayer 130 according to the present disclosure, the reaction temperature of the chamber 110 can be maintained by the heating of the microwave source 122. The water vapor of the reactant and the water resulted from the reaction will be further gasified due to the temperature and exhausted from the chamber 110 by using the vapor extractor 140. Accordingly, the present disclosure allows the reactant absorbing the microwaves uniformly so as to promote the reaction rate by the effect of microwave.

In order to maintain the temperature of the reactant in the containing space 111, the microwave reactor 100 can further include at least one heating source 121 connected to the side wall of the containing space 111 of the chamber 110. In FIG. 1, the heating source 121 is a jacketed pipe with a hot medium inside. Accordingly, the reactant within the containing space 111 can be maintained at a predetermined temperature so that a significant difference between the temperature of the mist, which is sprayed by the sprayer 130, and the reaction temperature will be avoided. Moreover, the heating source 121 also can be but not limited to a microwave heating source.

In details, the sprayer 130 includes a conduit 131, a presser 132 and a nozzle 133. The conduit 131 is communicated with the containing space 111 of the chamber 110 for conducting the reactant into the sprayer 130. The presser 132, such as a pressurized motor, is connected between the conduit 131 and the nozzle 133 for increasing the pressure at the nozzle 133 when the reactant within the conduit 131 is dispensed. The nozzle 133 is disposed in the reacting space 112, and the reactant will be dispensed through the nozzle 133 to become a mist. Then, the water contained in the mist will be gasified by the heating of the microwave source 122 for producing the water vapor. Accordingly, the above-mentioned problems, such as the penetration of the microwaves for the liquid reactant in the chamber and the heated uniformity of the reactant, can be solved.

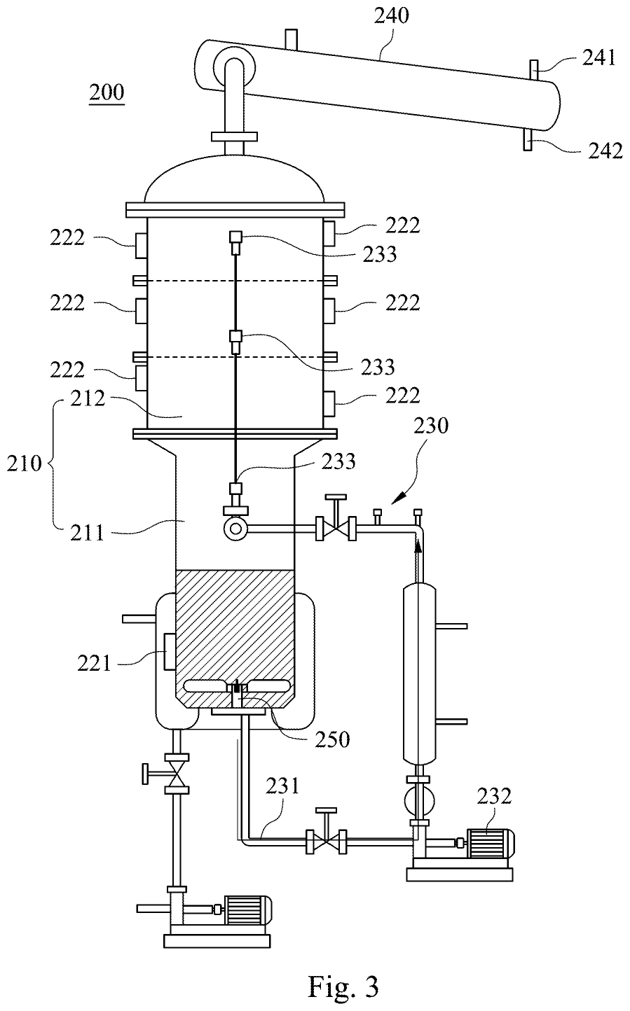

FIG. 3 is a schematic view of a microwave reactor 200 according to another embodiment of the present disclosure. As shown in FIG. 3, the microwave reactor 200 includes a chamber 210, at least one microwave source 222, a sprayer 230, a vapor extractor 240 and a stirring device 250.

The chamber 210 includes a containing space 211 and a reacting space 212, in which the containing space 211 is communicated with the reacting space 212 and for containing a reactant therein. Furthermore, an inner diameter of the reacting space 212 is larger than an inner diameter of the containing space 211 as shown in FIG. 3.

The microwave source 222 is connected to one side wall of the reacting space 212 of the chamber 200. In FIG. 3, there are a plurality of the microwave sources 222. Part of the microwave sources 222 are disposed corresponding to each other and located at the same heights of the side wall of the reacting space 212. However, another part of the microwave sources 222 are located at different heights of the side wall of the reacting space 212.

The sprayer 230 is for turning the reactant into the mist and spraying the mist in the reacting space 212. In details, the sprayer 230 includes a conduit 231, a presser 232 and at least one nozzle 233. The conduit 231 is communicated with the containing space 211 of the chamber 210 for conducting the reactant into the sprayer 230. The presser 232, such as a pressurized motor, is connected between the conduit 231 and the nozzle 233 for increasing the pressure at the nozzle 233 when the reactant within the conduit 231 is dispensed. The nozzle 233 is disposed in the reacting space 212, and the reactant will be dispensed through the nozzle 233 to become a mist. Then, the water contained in the mist will be gasified by the heating of the microwave source 222 for producing the water vapor. In detail, there are three nozzles arranged in sequence along an axial direction of the reacting space 212 in FIG. 3. Accordingly, more reactants can be sprayed at the same time for improving the yield.

The vapor extractor 240 is connected to the reacting space 212. When the water contained in the mist is gasified in the reacting space 212 to produce a water vapor, the water vapor can be exhausted from the chamber 210 by the vapor extractor 240. In details, the vapor extractor 240 includes a vacuum pump 241 and a condensed water outlet 242. The water vapor can be extracted by using the vacuum pump 241 and further directed outward through the condensed water outlet 242. Moreover, the water vapor also can be produced by gasifying water generated after the mist undergoes a chemical reaction.

The stirring device 250 is disposed in the containing space 211 of the chamber 210 for stirring the reactant.

In order to maintain the temperature of the reactant in the containing space 211, the microwave reactor 200 can further include at least one heating source 221. The heating source 221 is connected to the side wall of the containing space 211 of the chamber 210. In FIG. 3, the heating source 221 is a jacketed pipe with hot medium inside. Accordingly, the reactant within the containing space 211 can be maintained at a predetermined temperature so that a significant difference between the temperature of the mist, which is sprayed by the sprayer 230, and the reaction temperature will be avoided.

When the microwave reactor 200 is applied for the manufacturing process of the biodiesel, as the same as the microwave reactor 100 of FIG. 1, the reactant can be atomized and spayed in the reacting space 212 through the sprayer 230. Then, the reaction temperature of the chamber 210 can be maintained by the heating of the microwave source 222. At that time, the water contained in the reactant will be gasified due to the high temperature of the chamber 210 and exhausted from the chamber 210 by using the vapor extractor 240. Accordingly, the present disclosure allows the reactant absorbing the microwaves uniformly so as to promote the reaction rate by the effect of microwave.

Furthermore, through the configuration of the stirring device 250 at the bottom of the containing space 211 of the chamber 210, a heated uniformity of the reactant can be improved by stirring.

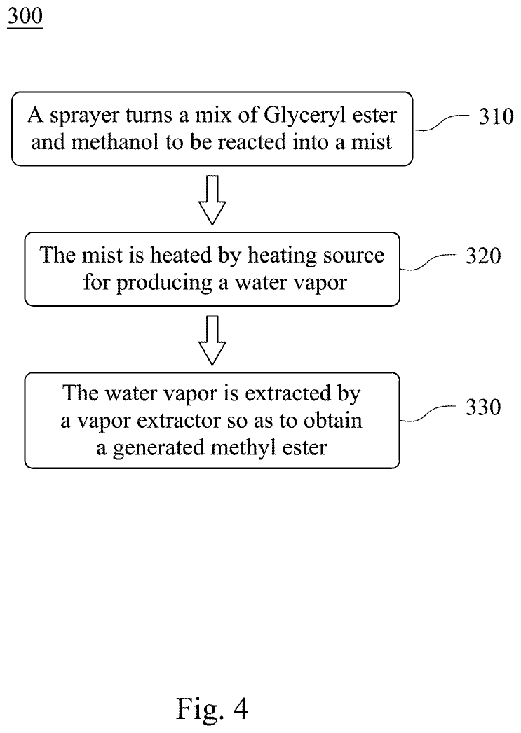

FIG. 4 is a flow chart of a manufacturing method 300 of biodiesel according to yet another embodiment of the present disclosure. In FIG. 4, the manufacturing method 300 of biodiesel includes the following steps and can be performed in coordination with the microwave reactor 100 of FIG. 1. However, the present disclosure is not limited thereto.

In Step 310, the sprayer 130 turns a mix of Glyceryl ester and methanol to be reacted into a mist at first and sprays the mist in the reacting space 112.

In Step 320, the mist is heated by the at least one heating source 122 so as to gasify the water contained for producing a water vapor.

In Step 330, the water vapor is extracted by the vapor extractor 140 so as to obtain a generated methyl ester, that is, a biodiesel.

As mentioned above, the microwaves cannot penetrate the large volume reactant to promote the reaction inside a large chamber, which contains the mix of Glyceryl ester and methanol to be reacted, is directly heated without the atomization of the reactants to be reacted so that it is unfavorable for controlling the reaction rate and the quality of the biodiesel. However, by the application of the above-mentioned steps of the manufacturing method 300 of biodiesel, such the problem can be solved.

Moreover, the mix of Glyceryl ester and methanol to be reacted can be maintained at a predetermined temperature through the at least one heating source 121. Accordingly, it is favorable for maintaining the temperature of the mix of Glyceryl ester and methanol to be reacted before the atomization so as to avoid the significant difference between the temperature of the mist and the reaction temperature.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A microwave reactor, comprising:
a chamber comprising a containing space and a reacting space, wherein the containing space is communicated with the reacting space and for containing a reactant therein;
at least one microwave source connected to one side wall of the reacting space of the chamber;
a sprayer communicated with the containing space of the chamber for turning the reactant into a mist and spraying the mist in the reacting space of the chamber; and
a vapor extractor connected to the reacting space;
wherein when a water contained in the mist is gasified in the reacting space to produce a water vapor, the water vapor is allowed to be exhausted from the chamber by the vapor extractor.

2. The microwave reactor of claim 1, wherein a number of the at least one microwave source is two.

3. The microwave reactor of claim 2, wherein the two microwave sources are correspondingly disposed to the side wall of the reacting space.

4. The microwave reactor of claim 1, wherein there are a plurality of the microwave sources located at the same heights of the side wall of the reacting space.

5. The microwave reactor of claim 1, wherein there are a plurality of the microwave sources located at different heights of the side wall of the reacting space.

6. The microwave reactor of claim 1, further comprising:
at least one heating source connected to the side wall of the containing space of the chamber.

7. The microwave reactor of claim 6, wherein the at least one heating source is a jacketed pipe with a hot medium inside or a microwave heating source.

8. The microwave reactor of claim 1, further comprising:
a stirring device disposed in the containing space of the chamber.

9. The microwave reactor of claim 1, wherein the sprayer comprising:
a conduit communicated with the containing space of the chamber;
a presser; and
at least one nozzle located in the reacting space of the chamber, wherein the presser is connected between the conduit and the at least one nozzle.

10. The microwave reactor of claim 9, wherein a number of the at least one nozzles is three.

11. A manufacturing method of a biodiesel, comprising:
turning a mix of Glyceryl ester and methanol to be reacted into a mist through a sprayer and spraying the mist in a reacting space;
heating the mist by at least one heating source for gasifying a water contained in the mist to produce a water vapor; and
extracting the water vapor by a vapor extractor and obtaining a generated methyl ester.

12. The manufacturing method of claim 11, wherein the mix of Glyceryl ester and methanol to be reacted is maintained at a predetermined temperature by at least one heating source.
